(19) United States
(12) Patent Application Publication
(10) Pub. No.: US 2023/0094001 A1
(43) Pub. Date: Mar. 30, 2023

(54) Prismatic Triangulating Corneal Topography System and Methods of Use

(71) Applicant: Tracey Technologies Corp., Houston, TX (US)

(72) Inventors: Paul van Saarloos, Balcatta (AU); Jinfeng Chen, Houston, TX (US)

(73) Assignee: Tracey Technologies Corp., Houston, TX (US)

(21) Appl. No.: 17/926,610

(22) PCT Filed: May 20, 2021
(86) PCT No.: PCT/US2021/033485

§ 371 Date: Nov. 20, 2022

Related U.S. Application Data
(60) Provisional application No. 63/027,747, filed on May 20, 2020.

(51) Int. Cl. A61B 3/107; A61B 3/14; A61B 3/00

(52) U.S. Cl. CPC A61B 3/107; A61B 3/145; A61B 3/0041

(57) ABSTRACT

Provided herein is a corneal topography system that utilizes a prism placed in optical alignment between the pattern image generator, such as a Placido disk, and the eye. The corneal topography system may be a prismatic triangulating corneal topography system that utilizes light rays of angle θ at the edge of the prism not passing through the prism, light rays that deviate from angle θ passing through the prism and light rays of angle a calculated from the reflection image to determine the corneal reflection point on the corneal surface. Also provided is a method for mapping a corneal surface of an eye of a subject utilizing an optical prism to produce a reflection image from a corneal surface reflection point on the corneal surface of the eye.

### CROSS-REFERENCE TO RELATED APPLICATIONS

This international application claims benefit of priority under 35 U.S.C. § 119(e) of provisional application U.S. Ser. No. 63/027,747, filed May 20, 2020, the entirety of which is hereby incorporated by reference.

### BACKGROUND OF THE INVENTION

### Field of the Invention

This invention relates to ophthalmic instruments. More specifically, the present invention is directed to an improved system and method for measuring corneal curvature and topography by capturing Placido disk images reflected in the cornea.

### Description of the Related Art

Measuring the topography of the corneal surface has become an important part of ophthalmology. It is used to detect disorders like keratoconus and to assess an eye's suitability for and to plan for surgery, for example, LASIK, or the use of premium intra-ocular lenses during cataract surgery.

There are currently two distinct technologies available for measuring corneal topography. One of them is the slit projection system, which involves projecting slits of light into the eye and capturing an image of the eye from an angle relative to the direction of the light slit projection. The second involves imaging targets reflected onto the corneal surface. The target is usually a placido disk which is made up of black and white concentric rings.

Slit projection systems measure the posterior surface of the cornea. However, the method requires multiple measurements using different slit orientations/positions to get the entire topography, which is a disadvantage since eye movement during measurements can potentially compromise accuracy. Moreover, current research suggests that the optical effect of eye to eye variations may not be clinically relevant.

By virtue of the reflection angle being twice the slope angle of the reflecting surface, reflection-based topography systems can be extremely precise. However, calculating the shape of the corneal surface from the image reflected onto it is non-trivial and may not have a unique solution. One such calculation method is described by van Saarloos et al. (1). van Saarloos discloses a mathematical method for estimating the central corneal radius of curvature and for calculating corneal topography from the radii of the rings in a placido disk image. This method, as well as similar methods of calculation, require the distance between the eye and the topography device to be accurately known and for the eye shape to match the assumptions of shape used in the calculation methods.

Accuracy of topography systems that rely on the user to accurately position the device relative to the eye depend heavily on operator skills. Delays caused by repeated alignment for optimization can cause the eye to dry, creating an inaccurate measurement, adding to operating costs.

U.S. Pat. No. 5,418,582 teaches a system and method where the placido disk has an added ring or point light source well outside the plane formed by the adjacent rings. This allows parallax to be used to calculate the distance between the device and the eye and greatly simplifies use of the topographer since it removes user error, thereby allowing quicker measurements of angle α from an image of the reflected rings. However, this improvement still relies on the assumptions of shape (corneal topography) for applying the calculations. Specifically, without prior knowledge on how far the corneal surface is from the topography device, there are many possible solutions for corneal topography. Three possible exemplary solutions are shown, i.e., a steep corneal surface closer to the topographer, a flat corneal surface further away from the device, and a medium curvature in between.

Overall, therefore, there is a deficiency in the art for optimal topography systems and methods, which improve accuracy while limiting or eliminating the need for making assumptions of shape. The present invention fulfills this longstanding need and desire in the art.

### SUMMARY OF THE INVENTION

The present invention is directed to a corneal topography system for mapping a corneal surface of an eye in a subject. The system has at least pattern image generator and at least one optical prism in optical alignment between the pattern image generator and the corneal surface of the eye for which topographical information is desired. A light source is disposed in optical alignment with the pattern image generator and an image sensor is disposed in optical alignment with the corneal surface of the eye. A means for electronically transmitting data from the image sensor to an electronic device is configured to analyze the data and to display results of the analysis.

The present invention is directed to a related corneal topography system further comprising a focusing lens placed between the image sensor and the corneal surface. The present invention is directed to another related corneal topography system further comprising a pattern image generator comprising a checkerboard pattern disposed thereon.

The present invention also is directed to a prismatic triangulating corneal topography system for mapping a corneal surface of an eye. The system has at least one Placido disk and at least one prism in optical alignment between the Placido disk and the corneal surface of the eye for which topographical information is desired. A light source is disposed in optical alignment with the Placido disk and an image sensor is disposed in optical alignment with the corneal surface of the eye. An electronic device comprising image analysis software tangibly stored therein is in electronic communication with the image sensor.

The present invention is directed to a related prismatic triangulating corneal topography system further comprising an optical lens disposed between the image sensor and the corneal surface. The present invention is directed to a related prismatic triangulating corneal topography system further comprising a Placido disk comprising a black and white checkerboard pattern disposed on an outer ring thereof.

The present invention is directed further to a method for mapping a corneal surface of an eye of a subject. An optical prism is positioned between a Placido disk and the corneal surface of the eye of the subject in a corneal topography system and the Placido disk and the optical prism are illuminated to generate a ring pattern therefrom. A reflection of the Placido disk from the corneal surface of the eye generated by the illuminating step is acquired with an image sensor where the reflection originates from a corneal surface reflection point on the corneal surface of the eye. The reflection image is transmitted from the image sensor to a computer to measure at least one parameter of the corneal surface and the at least one parameter is mapped to produce a corneal topography map of the eye.

The present invention is directed to a related method for mapping a corneal surface of an eye of a subject further comprising displaying the corneal topography map on the computer. The present invention is directed to another related method further comprising calculating angle α from the reflection image acquired by the image sensor, wherein a light ray at the angle α intersects a light ray with angle θ from beside the Placido ring at the corneal reflection point on the corneal surface. The present invention is directed to yet another related method further comprising measuring the deviation of the ring pattern from light rays at the angle θ beside the prism. The present invention is directed to yet another related method further comprising calculating a surface tangent angle at the corneal surface reflection point from the angle α and the angle θ. The present invention is directed to yet another related method further comprising minimizing an estimation of the working distance between a corneal apex and the image sensor.

Other and further aspects, features, benefits, and advantages of the present invention will be apparent from the following description of the presently preferred embodiments of the invention given for the purpose of disclosure.

### BRIEF DESCRIPTION OF THE DRAWINGS

FIG. 1A is a cross-sectional view of a standard placido disk corneal topography system.

FIG. 1B is a cross sectional view of a prismatic corneal topography system showing an optical prism placed between the pattern image generator and the eye.

FIG. 2 illustrates the refraction of the Placido disk rings through one optical prism.

FIG. 3 is a cross-sectional view of the corneal topography system with an additional prism.

FIG. 4A is the reflection of a traditional Placido disk on the eye.

FIG. 4B is the detected edge of the rings of the Placido disk in FIG. 4A.

FIG. 4C is the reflection of the Placido disk on the eye produced by the corneal topography system showing the four wedges encompassing the rings' edges.

FIG. 4D illustrates with outlines the ring's edges of FIG. 4B detected by the traditional method.

FIG. 5 is a Placido disk with a white and black checkerboard pattern on the outer ring.

### DETAILED DESCRIPTION OF THE PRESENT INVENTION

For convenience, before further description of the present invention, certain terms employed in the specification, examples and appended claims are collected herein. These definitions should be read in light of the remainder of the disclosure and understood as by a person of skill in the art. Unless defined otherwise, all technical and scientific terms used herein have the same meaning as commonly understood by a person of ordinary skill in the art.

As used herein, “a” or “an” may mean one or more. As used herein in the claim(s), when used in conjunction with the word “comprising”, the words “a” or “an” may mean one or more than one.

As used herein, the term “or” in the claims is used to mean “and/or” unless explicitly indicated to refer to alternatives only or the alternatives are mutually exclusive, although the disclosure supports a definition that refers to only alternatives and “and/or.”

As used herein “another” or “other” may mean at least a second or more of the same or different claim element or components thereof. Similarly, the word “or” is intended to include “and” unless the context clearly indicates otherwise.

As used herein, “comprise” or “comprises” or “comprising”, except where the context requires otherwise due to express language or necessary implication, are used in an inclusive sense, i.e. to specify the presence of the stated features but not to preclude the presence or addition of further features in various embodiments of the invention.

As used herein, “including”, “which includes” or “that includes” is also an open term that also means including at least the elements/features that follow the term, but not excluding others. Thus, including is synonymous with and means comprising.

As used herein, the ordinal adjectives “first” and “second”, unless otherwise specified are used to describe a common object, merely indicate that different instances of like objects are being referred to, and are not intended to imply that the objects so described must be in a given sequence, either temporally, spatially, in ranking, or in any other manner. Moreover, as used herein, “first optical prism” and “first prism” are interchangeable and “second optical prism” and “second prism”, are interchangeable.

As used herein, the terms “optical prism”, “prism” are used interchangeably and refer to an optical element that refracts light.

In one embodiment of this invention, there is provided a corneal topography system for mapping a corneal surface of an eye in a subject, comprising at least one pattern image generator; at least one optical prism disposed in optical alignment between the pattern image generator and the corneal surface of the eye for which topographical information is desired; a light source disposed in optical alignment with the pattern image generator; an image sensor disposed in optical alignment with the corneal surface of the eye; and means for electronically transmitting data from the image sensor to an electronic device configured to analyze the data and to display results of the analysis.

Further to this embodiment the corneal topography system comprises a focusing lens disposed between the image sensor and the corneal surface. In another further embodiment the corneal topography system comprises a pattern image generator comprising a checkerboard pattern disposed thereon. In this further embodiment the image generator may be a Placido disk comprising a black and white checkerboard pattern disposed on an outer ring thereof.

In all embodiments the pattern image generator may comprise alternating opaque and transparent concentric rings. Also, the optical prism may be a triangular prism, or a cuboid prism, or a hexagonal prism. In addition the image sensor may be a charge-coupled device or a complementary metal-oxide semiconductor.

In another embodiment of the present invention there is provided a prismatic triangulating corneal topography system for mapping a corneal surface, comprising at least one Placido disk; at least one optical prism disposed in optical alignment between the Placido disk and the corneal surface of the eye for which a topographical information is desired; a light source disposed in optical alignment with the Placido disk; an image sensor disposed in optical alignment with the corneal surface of the eye; and an electronic device with image analysis software tangibly stored therein in electronic communication with the image sensor.

Further to this embodiment the prismatic triangulating corneal topography system comprises an optical lens disposed between the image sensor and the corneal surface. In another further embodiment the prismatic triangulating corneal topography system comprises a Placido disk comprising a black and white checkerboard pattern disposed on an outer ring thereof.

In all embodiments the optical prism may be a triangular prism. Also the image sensor may be a charge-coupled device or a complementary metal-oxide semiconductor. In addition the electronic device may be a desktop computer, a laptop computer, or a smart device.

In yet another embodiment of the present invention there is provided method for mapping a corneal surface of an eye of a subject, comprising positioning an optical prism between a Placido disk and the corneal surface of the eye of the subject in a corneal topography system; illuminating the Placido disk and the optical prism to generate a ring pattern therefrom; acquiring with an image sensor a reflection of the Placido disk from the corneal surface of the eye generated by the illuminating step, said reflection originating from a corneal surface reflection point on the corneal surface of the eye; transmitting the reflection image from the image sensor to a computer to measure at least one parameter of the corneal surface; and mapping the at least one parameter to produce a corneal topography map of the eye.

Further to this embodiment the method comprises displaying the corneal topography map on the computer. In these embodiments the optical prism may be positioned such that the Placido disk is seen in the reflection image through the optical prism and on both sides of the edge of the prism.

Also in these embodiments at the edge of the optical prism, a deviation of the ring pattern from an angle θ looking through the optical prism compared to the ring pattern at the angle θ looking beside the optical prism provides a line of sight from which the ring pattern is viewed. Further to this embodiment the method comprises calculating angle α from the reflection image acquired by the image sensor, wherein a light ray at the angle α intersects a light ray with angle θ from beside the Placido ring at the corneal reflection point on the corneal surface. In a further aspect of these embodiments the method comprises measuring the deviation of the ring pattern from light rays at the angle θ beside the prism. In another further aspect the method comprises calculating a surface tangent angle at the corneal surface reflection point from the angle α and the angle θ.

In yet another further embodiment the method comprises minimizing an estimation of the working distance between a corneal apex and the image sensor. In this further embodiment the method may comprise the steps of determining a radius of the corneal surface of the eye; positioning a Placido disk comprising a black and white checkerboard pattern disposed on an outer ring thereof such that an image of the checkerboard Placido disk is reflected on the corneal surface upon illumination thereof; determining the magnification of the imaging sensor; measuring the widths of the black blocks and the white blocks at a plurality of points as the cornea is moved closer to and farther away from the imaging sensor; and calculating an average working distance based on the magnification of the imaging sensor and the measured widths of the black blocks and the white blocks of the checkerboard.

In all embodiments and further aspects thereof the parameter may comprise position, elevation or slope. Also the optical prism may be a triangular prism, a cuboidal prism, or a hexagonal prism. In addition the image sensor may be a charge-coupled image sensor or a complementary metal-oxide semiconductor image sensor.

Provided herein is a corneal topography system that utilizes at least one optical prism, for example, but not limited to, a first optical prism or first prism and a second optical prism or second prism, disposed between a means to generate a pattern, such as, but not limited to, a pattern image generator, for example, a Placido disk, and the corneal surface of the eye. The corneal topography system may be a prismatic triangulating corneal topography system in which light rays refracted through the prism, light rays bypassing the prism and light rays with angle α calculated from the reflection image enable the corneal reflection point to be triangulated and its position on the corneal surface identified.

The optical prism may be any type of prism including, but not limited to, a triangular prism, a cuboidal prism and a hexagonal prism. The prism may be manufactured using any optically transparent material including, but not limited to glass, quartz and fluorite.

The prism is positioned with respect to the pattern image generator such that a portion of the light source passing through the pattern image generator is deviated by refraction through the prism before being incident on the corneal surface, and another portion of the light source passing through the pattern image generator is directly incident on the corneal surface. The prism is oriented such that its edge is perpendicular to the rings in the pattern image generator. Thus, the apparent deviation of the ring enabled by the prism provides a line from which the ring is viewed. Alternatively, multiple prisms, with their edges aligned perpendicular to the rings, may be used. A large number of reflection points on the corneal surface can have their exact topography parameters calculated accurately, allowing assumptions in the corneal shape to be largely eliminated.

This is advantageous over standard corneal topographers including a Placido disk topographer where a point in the image of the pattern reflected in the corneal surface provides a line in which the corneal reflection point must be on, but cannot differentiate between a flat corneal surface further away or a steeper corneal surface closer. Thus, the present invention provides a unique solution for the reflection point on the cornea, both for position, including elevation, and surface slope. In addition, these prisms allow a quick and simple point and shoot measurement process, that does not need any special alignment.

The light source illuminates the image pattern generator and is in optical alignment with the pattern image generator and the prism. The light source may be any light source suitable for corneal topography as is known and standard in the art. For example, monochrome light may be more accurate than a broad spectrum source

The corneal topography system comprises an image sensor that captures an image of the pattern reflected from the corneal surface of the eye. Any commercially available stand-alone image sensors comprised in a digital camera or a smart device or, image sensors integrated with a computer for image analysis may be used for this purpose Examples of such image sensors include a charge-coupled device (CCD) and an active-pixel sensor (CMOS sensor).

The corneal topography system comprises a means for focusing an image of the pattern reflected from the corneal surface on the image sensor before the image is digitally captured by the image sensor. For example a focusing lens or an optical lens is disposed in optical alignment between the image sensor and the corneal surface. Any commercially available lens may be utilized to serve as the focusing lens or the optical lens. The lens may be made using any optically transparent material including, but not limited to, glass, quartz and plastic.

The corneal topography system may comprise a Placido disk with a ring checkerboard pattern (ring checkerboard Placido disk). The corneal radius may be determined or calculated using the ring algorithm. The checkerboard ring on the disk can be used to minimize the working distance estimation by averaging the distance, where the working distance is the distance between the corneal apex to the CCD camera. The working distance may be calculated using the checkerboard via two methods. The first method comprises calculating the magnification of the camera system. The desired working distance can be solved using the algorithm developed for the camera magnification. The width of the black and white blocks in the checkerboard increases as the cornea is moved closer to the camera and decreases as the cornea is moved farther away from the camera. In the second method the working distance is empirically determined by physically measuring the widths of the white blocks and the widths of the black block at different distances. Using two measured points enables the equation of the line to be developed.

The corneal topography system is electronically linked to means for electronically transmitting data from the image sensor to an electronic device configured to analyze the data and to display results of the analysis. The means may comprise any type of commercially available electronic device with a processor, a memory, display, and at least one network connection may be used. These devices include, but not limited to, a desktop computer, a laptop computer, a hand-held computer or tablet, and a smart device. The electronic device is in wired or wireless communication with the image sensor. The electronic device tangibly stores that software, firmware and/or algorithms suitable for analysis of the captured reflection image as is known and standard in the art.

Also provided is a method for mapping a corneal surface of an eye of a subject that utilizes the corneal topography systems described herein. Particularly, the optical prism is placed between a Placido disk and the corneal surface of the eye. A subject for which topographical information of the corneal surface of the eye is desired sees the rings of the Placido disk through the prism and the rings at both sides of the prism. By placing the optical prism at this position, two light paths are generated—a deviated light that is refracted through the prism and a non-deviated light that does not pass through the prism. Both light rays or light paths are incident upon the corneal surface to generate a reflection pattern image that when captured by an image sensor provides a unique solution for a corneal surface reflection point. The method also enables angle α and angle θ to be calculated, the deviation from angle θ at the edge of the prism of the light path through the prism and the surface tangent angle or slope at the corneal surface reflection point.

The Placido disks in the present systems are configured to produce an image on the corneal surface showing wedges, for example, but not limited to, within four wedged sections. The wedges are equally spaced on the image and enable an improved edge detection.

Particularly, embodiments of the present invention are better illustrated with reference to the Figure(s), however, such reference is not meant to limit the present invention in any fashion. The embodiments and variations described in detail herein are to be interpreted by the appended claims and equivalents thereof.

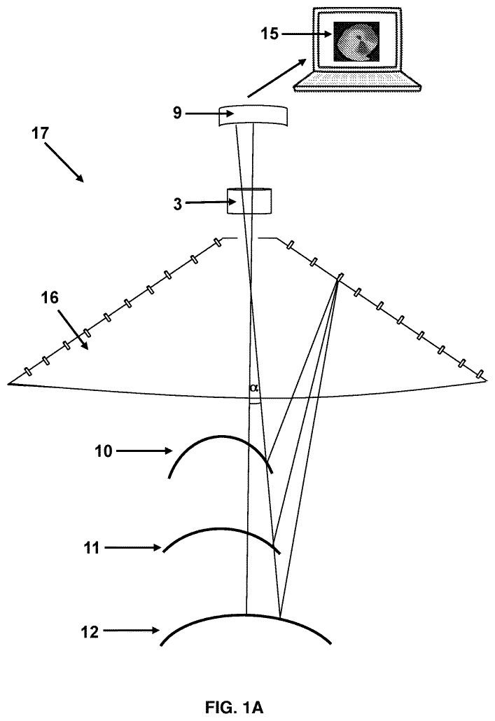

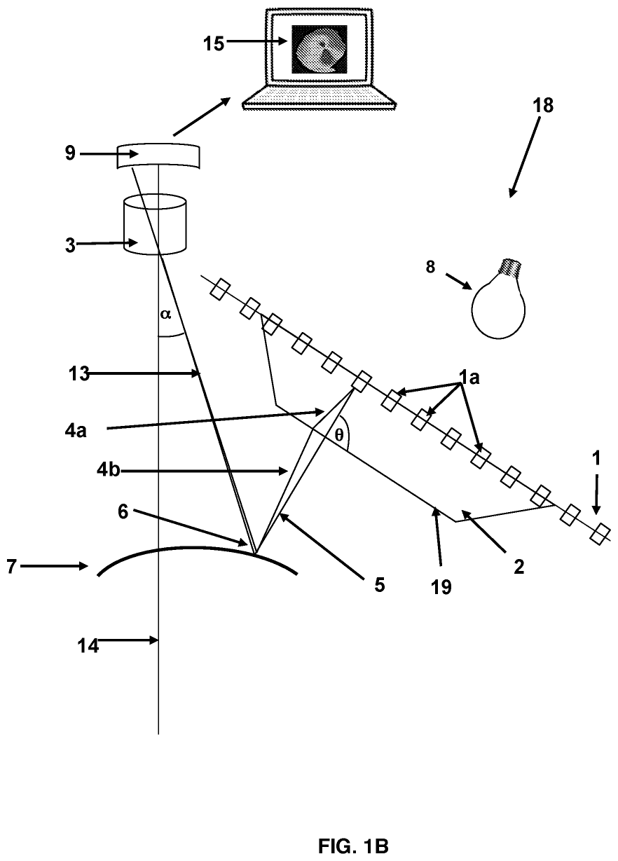

FIG. 1A shows a corneal topography system 17 as known in the art. The system consists of a cone-shaped pattern image generator 16 that generates an image on a corneal surface 10-12, a lens 3 that focuses the reflected light from the corneal surface to a charge-coupled device (CCD) 9, which serves as the image sensor. The image captured by the charge-coupled device is transmitted to a computer 15 for analysis. This system relies on the assumptions of corneal shape for applying the calculations. Without prior knowledge however on how far the corneal surface 10-12 is from the topography device, there are many possible solutions for corneal topography including, a steep corneal surface 10 closer to the topographer, a flat corneal surface 12 further away from the device and a medium curvature corneal surface 11 in between.

FIG. 1B is a cross sectional view of the corneal topography system 18 along an edge 19 of the prism 2 showing a light source 8, a pattern image generator 1 with a plurality of rings 1a disposed thereon and a lens 3. A ring image reflected off the corneal surface 7 passes through the lens and is captured by a charge-coupled device (CCD) 9 used as the image sensor. The CCD image captured by the charge-coupled device is transmitted to a computer 15 for analysis. From the image, a first angle θ is measured between the edge of the prism and the light source 5 not passing through the prism. A second angle α is measured between a visual axis 14 perpendicular to the corneal surface and the light source reflected 13 from the corneal surface. Since only light rays at angle θ along the edge of the prism will have the measured deviation when moving through the prism, the deviation can also be measured from the CCD image. The point of intersection of the light passing through 4a the prism and refracted 4b, and the non-deviated light path 5, which does not pass through the prism is the corneal surface reflection point 6. The two angles α and θ also enable the surface tangent angle at that surface reflection point to be easily calculated.

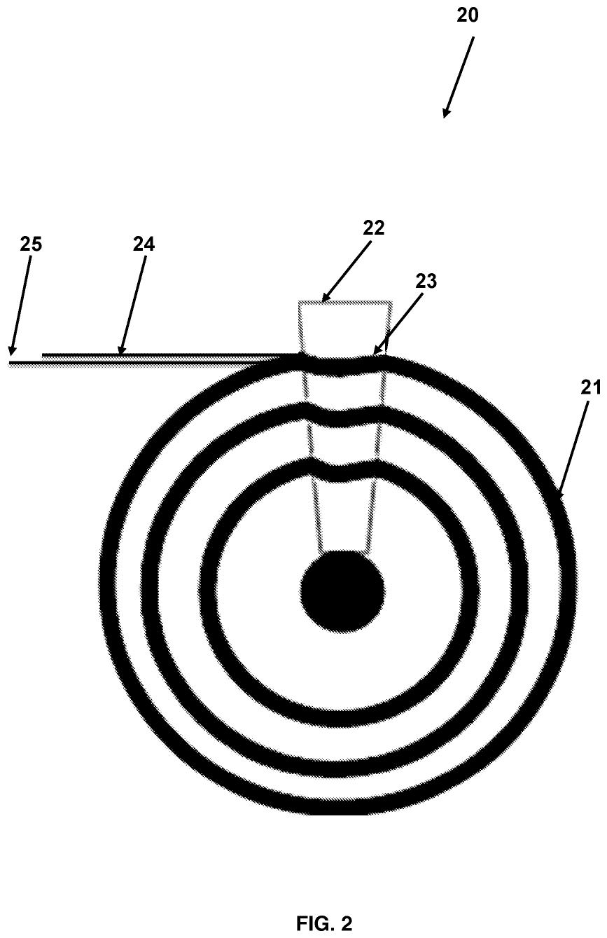

FIG. 2 shows a Placido disk 20 in a corneal topography system that utilizes one optical prism illustrating the refraction of the rings through one optical prism. The Placido disk contains three rings represented by 21. A four-sided prism 22 illustrates how the Placido disk rings are refracted through the prism at 23. The offset caused by the refraction between the disk rings and the prism is shown by 24,25.

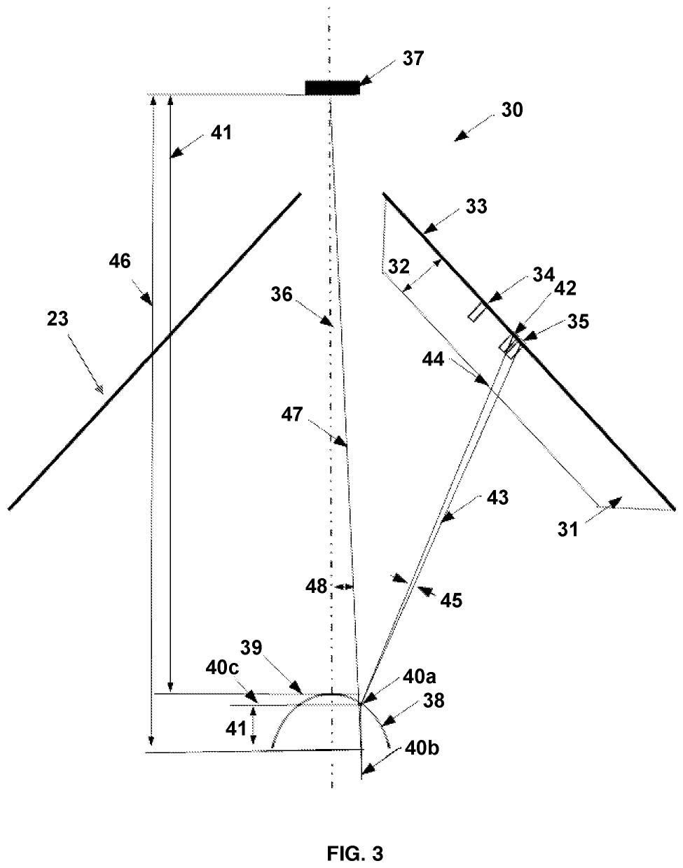

FIG. 3 is a cross-sectional view of the corneal topography system 30 with a second prism 31. In addition to the first prism 22, the second prism has a thickness 32 and index of refraction of prism “n”. A cone 33 with two rings 34,35 is centered along the optical axis 36 with the CCD camera 37. The cornea 38 also is centered with the optical axis. The corneal apex is shown at 39. The cone is reflected off the cornea point (xi,yi) at 40a,b,c. Cornea point (xi,yi) is referenced to the limbus plane 41. The cone ring 35 is displaced by the thickness and index of refraction of the prism as shown in 23 (see FIG. 3) and 42. The prism causes a ring displacement illustrated by light rays 43,44 and angle 45. The distance 46 between the camera and corneal apex 39 is determined using the angle formed between the optical axis and the light ray 47 to the corneal point (xi,yi) 40b. The working distance between the limbus plane and camera is determined from the corneal and angle 48.

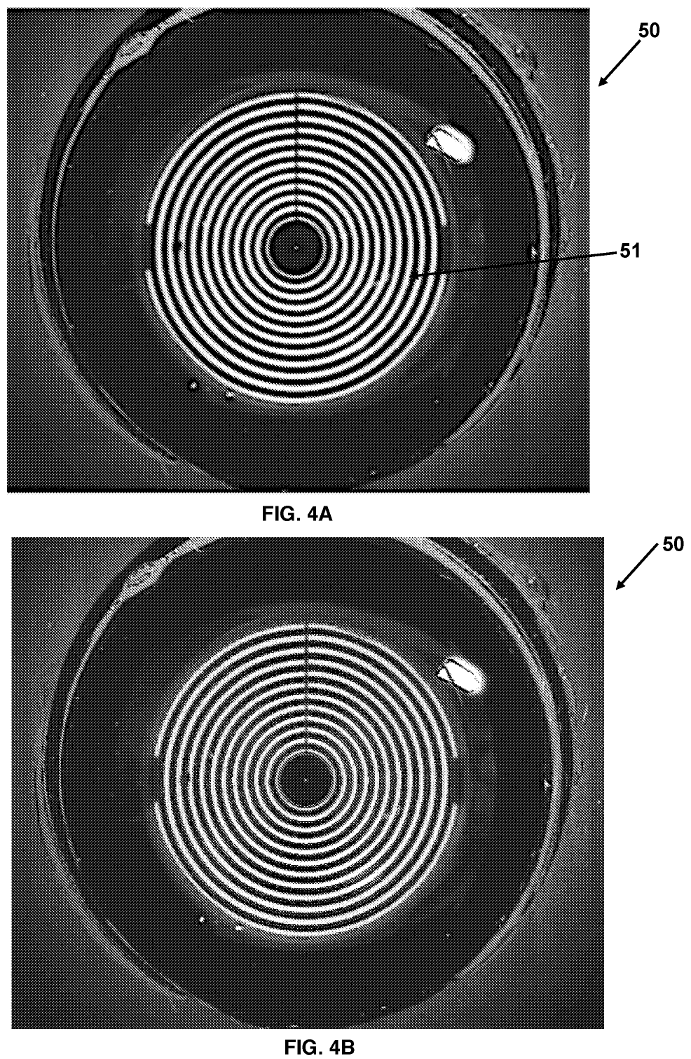

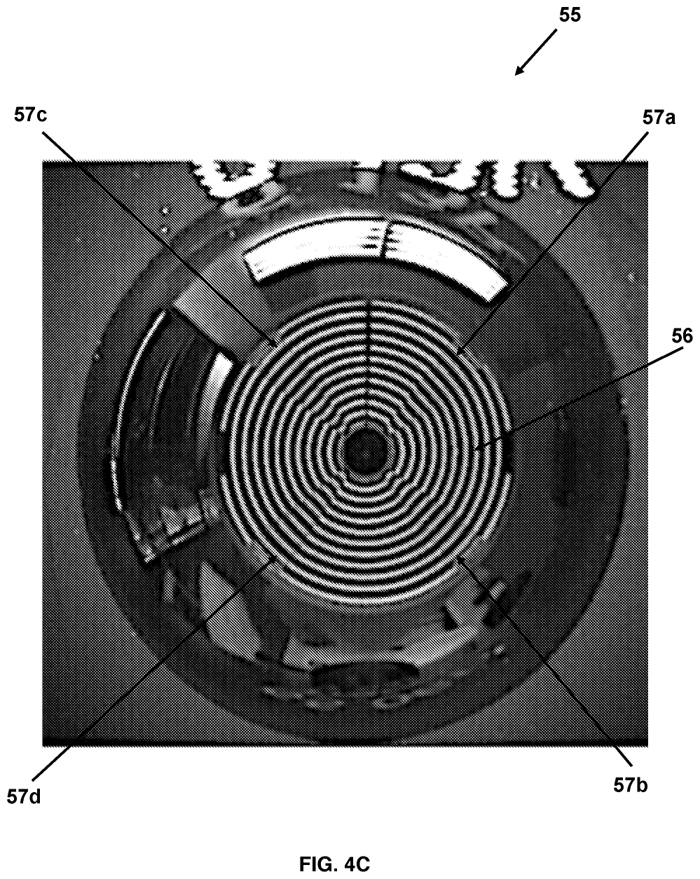

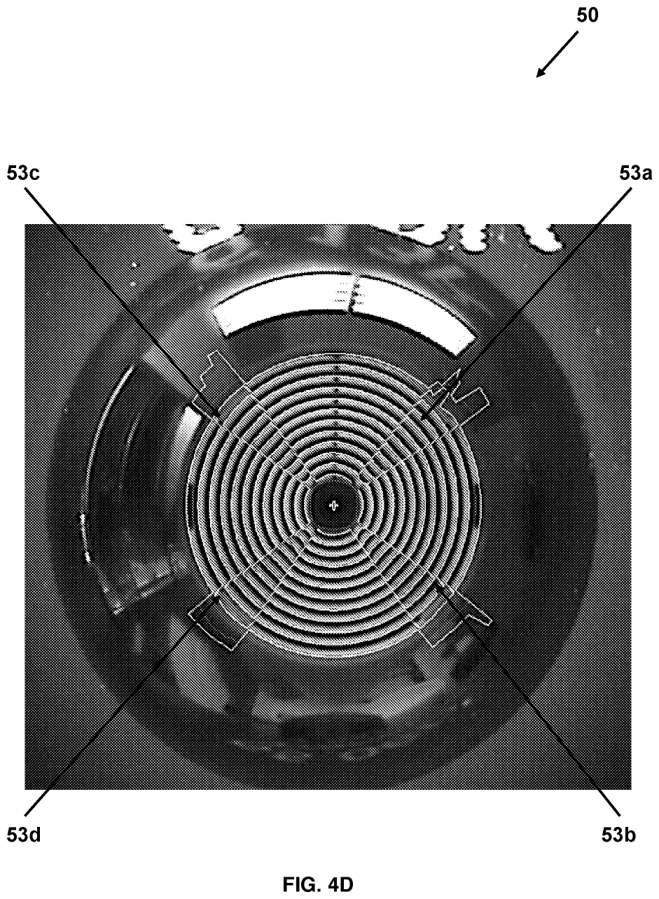

FIGS. 4A-4D compare reflection images 50 of a Placido disk produced by the traditional method to improved reflection images 55 of a Placido disk produced by the corneal topography system. FIG. 4A illustrates a traditional Placido disk 50 with the plurality of concentric rings represented by 51. With continued reference to FIG. 4A, FIG. 4B illustrates that the detected edges of the rings are difficult to visualize (see FIG. 4D). FIG. 4C illustrates the image of the Placido disk 55 with the plurality of concentric rings represented by 56 produced by the corneal topography system. The reflection image has four wedges 57a,b,c,d highlighting the detected rings' edges for improved visibility. With continued reference to FIG. 4B, FIG. 4D outlines at 53a,b,c,d the detected edges in FIG. 4B.

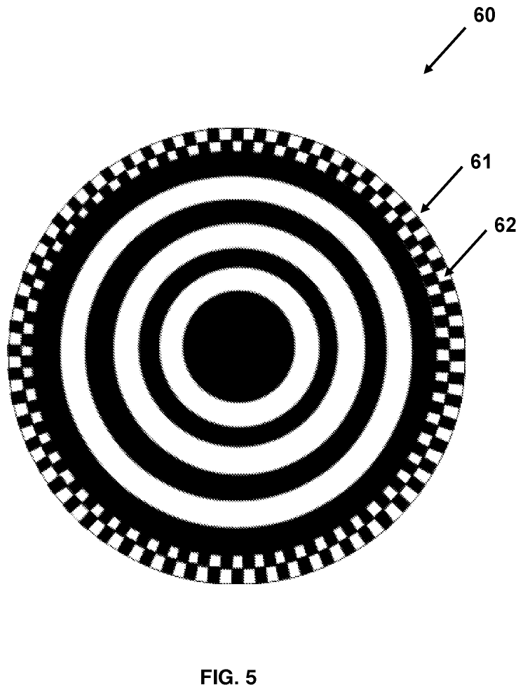

With continued reference to FIG. 3, FIG. 5 is a Placido disk 60 used to minimize the estimation of working distance between the corneal apex 39 to the CCD camera 37 (see FIG. 3). The Placido disk has an outer ring 61 with a checkerboard pattern 62 circumferentially disposed thereon.

The present invention is well adapted to attain the ends and advantages mentioned as well as those that are inherent therein. The particular embodiments disclosed above are illustrative only, as the present invention may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered or modified and all such variations are considered within the scope and spirit of the present invention. The terms in the claims have their plain, ordinary meaning unless otherwise explicitly and clearly defined by the patentee.

### THE FOLLOWING REFERENCE IS CITED HEREIN

1. van Saarloos, P P and Constable, I J. (1991) Optometry and Vision Science, 68 (12): 960-965.

What is claimed is:

1. A corneal topography system for mapping a corneal surface of an eye in a subject, comprising:
at least one pattern image generator;
at least one optical prism disposed in optical alignment between the pattern image generator and the corneal surface of the eye for which topographical information is desired;
a light source disposed in optical alignment with the pattern image generator;
an image sensor disposed in optical alignment with the corneal surface of the eye; and
means for electronically transmitting data from the image sensor to an electronic device configured to analyze the data and to display results of the analysis.

2. The corneal topography system of claim 1, further comprising a focusing lens disposed between the image sensor and the corneal surface of the eye.

3. The corneal topography system of claim 1, further comprising a pattern image generator comprising a checkerboard pattern disposed thereon.

4. The corneal topography system of claim 3, wherein the image generator is a Placido disk comprising a black and white checkerboard pattern disposed on an outer ring thereof.

5. The corneal topography system of claim 1, wherein the pattern image generator comprises alternating opaque and transparent concentric rings.

6. The corneal topography system of claim 1, wherein the optical prism is a triangular prism, or a cuboid prism, or a hexagonal prism.

7. The corneal topography system of claim 1, wherein the image sensor is a charge-coupled device or a complementary metal-oxide semiconductor.

8. A prismatic triangulating corneal topography system for mapping a corneal surface of an eye, comprising:
at least one Placido disk;
at least one prism disposed in optical alignment between the Placido disk and the corneal surface of the eye for which topographical information is desired;
a light source disposed in optical alignment with the Placido disk;
an image sensor disposed in optical alignment with the corneal surface of the eye; and
an electronic device comprising image analysis software tangibly stored therein in electronic communication with the image sensor.

9. The prismatic triangulating corneal topography system of claim 8, further comprising an optical lens disposed between the image sensor and the corneal surface of the eye.

10. The prismatic triangulating corneal topography system of claim 8, further comprising a Placido disk comprising a black and white checkerboard pattern disposed on an outer ring thereof.

11. The prismatic triangulating corneal topography system of claim 8, wherein the prism is a triangular prism.

12. The prismatic triangulating corneal topography system of claim 8, wherein the image sensor is a charge-coupled device or a complementary metal-oxide semiconductor.

13. The prismatic triangulating corneal topography system of claim 8, wherein the electronic device is a desktop computer, a laptop computer, or a smart device.

14. A method for mapping a corneal surface of an eye of a subject, comprising:
positioning an optical prism between a Placido disk and the corneal surface of the eye of the subject in a corneal topography system;
illuminating the Placido disk and the optical prism to generate a ring pattern therefrom;
acquiring with an image sensor a reflection of the Placido disk from the corneal surface of the eye generated by the illuminating step, said reflection originating from a corneal surface reflection point on the corneal surface of the eye;
transmitting the reflection image from the image sensor to a computer to measure at least one parameter of the corneal surface; and
mapping the at least one parameter to produce a corneal topography map of the eye.

15. The method of claim 14, further comprising displaying the corneal topography map on the computer.

16. The method of claim 14, wherein the optical prism is positioned such that the Placido disk is seen in the reflection image through the optical prism and on both sides of the edge of the prism.

17. The method of claim 14, wherein, at the edge of the optical prism, a deviation of the ring pattern from an angle θ looking through the optical prism compared to the ring pattern at the angle θ looking beside the optical prism provides a line of sight from which the ring pattern is viewed.

18. The method of claim 17, further comprising calculating angle α from the reflection image acquired by the image sensor, wherein a light ray at the angle α intersects a light ray with angle θ from beside the Placido ring at the corneal reflection point on the corneal surface.

19. The method of claim 18, further comprising measuring the deviation of the ring pattern from light rays at the angle θ beside the prism.

20. The method of claim 19, further comprising calculating a surface tangent angle at the corneal surface reflection point from the angle α and the angle θ.

21. The method of claim 19, further comprising minimizing an estimation of the working distance between a corneal apex and the image sensor.

22. The method of claim 21, comprising the steps of:
determining a radius of the corneal surface of the eye;
positioning a Placido disk comprising a black and white checkerboard pattern disposed on an outer ring thereof such that an image of the checkerboard Placido disk is reflected on the corneal surface upon illumination thereof;
determining the magnification of the imaging sensor;
measuring the widths of the black blocks and the white blocks at a plurality of points as the cornea is moved closer to and farther away from the imaging sensor; and
calculating an average working distance based on the magnification of the imaging sensor and the measured widths of the black blocks and the white blocks of the checkerboard.

23. The method of claim 14, wherein the parameter comprises position, elevation or slope.

24. The method of claim 14, wherein the optical prism is a triangular prism, a cuboidal prism, or a hexagonal prism.

25. The method of claim 14, wherein the image sensor is a charge-coupled image sensor or a complementary metal-oxide semiconductor image sensor.
